(19) United States
(12) Patent Application Publication
(10) Pub. No.: US 2022/0406433 A1
(43) Pub. Date: Dec. 22, 2022

(54) Matching Physical Rehabilitation Motions with a Gaming Experience Using Computer Vision on Mobile Phones

(71) Applicant: Reactiv Inc., Brooklyn, NY (US)

(72) Inventors: Arthur Sebert, Brooklyn, NY (US); Siddharth Saxena, Brooklyn, NY (US)

(21) Appl. No.: 17/843,487

(22) Filed: Jun. 17, 2022

Related U.S. Application Data
(60) Provisional application No. 63/211,862, filed on Jun. 17, 2021.

(51) Int. Cl. G16H 20/30; A63F 13/428

(52) U.S. Cl. CPC G16H 20/30; A63F 13/428; A63F 2300/60

(57) ABSTRACT

A physical rehabilitation tool has a personal electronic device with a processor, a camera and a display screen. The processor is programed to illustrate a game module on the display screen. The game module includes at least a first feature and a second feature. The camera is configured to superimpose an image of a body part over the game module. Movement of the image of the body part from the first feature to the second feature requires the body part to traverse a desired range of motion.

### CROSS-REFERENCE TO RELATED PATENT APPLICATIONS

This patent application claims a benefit to the Jun. 17, 2021, filing date of U.S. Provisional Patent Application Ser. No. 63/211,862 titled, “Matching Physical Rehabilitation Motions with a Gaming Experience Using Computer Vision on Mobile Phones.” The disclosure of U.S. 63/211,862 is incorporated by reference herein in its entirety.

### SUMMARY OF THE DISCLOSURE

The platform disclosed herein uses computer vision on mobile phones to detect rehab motions used for Occupational Therapy and match these motions with a gaming experience.

### BACKGROUND

Exercise feedback systems that receive exercise data captured by client devices of users performing musculoskeletal exercises and provide captured images to a physical trainer for feedback are disclosed in U.S. Pat. No. 10,902,741 B2, “Exercise Feedback System for Musculoskeletal Exercise” and United States Published Patent Application No. US 2019/0295438 A1, “Augmented Reality Guided Musculoskeletal Exercises,” both by Rubinstein et al.

### IN THE DRAWINGS

FIG. 1. illustrates a structure for an application disclosed herein.

FIG. 2 illustrates how a module, in particular a game, is built.

FIG. 3 illustrates one embodiment of detection algorithms to detect the medically correct motions for Range of Motion exercises.

FIG. 4 illustrates types of motion utilized by patients playing a game in accordance with the disclosure in bar graph format.

FIG. 5 illustrates the types of motion illustrated in FIG. 4 presented in pie chart format.

### DETAILED DESCRIPTION

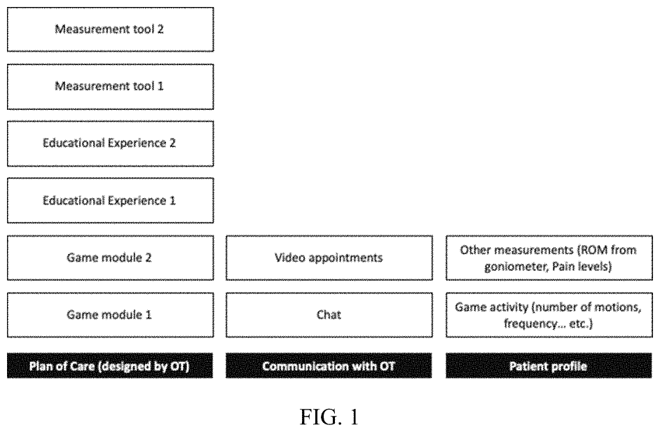

As shown in FIG. 1, the platform has three main parts:
1) The Plan of Care is a list of activities chosen by an occupational therapist (OT) who is following the patient. The activities are what the OT deems to be relevant modules for this patient: games, measurement tools, educational experiences. These modules are mainly for the patient to do on their own at home.
2) Communication with OT is where the patient talks to the OT via video (synchronous) or chat (asynchronous). Modules from the Plan of Care can be used during video chat for live assessment and feedback.
3) The Patient Profile is where the data from games and measurement tools is gathered. Both the patient and the therapist have access to it to follow the progress. The therapist may use this data to adjust the Plan of Care as they see fit.

### Game Design and Engineering

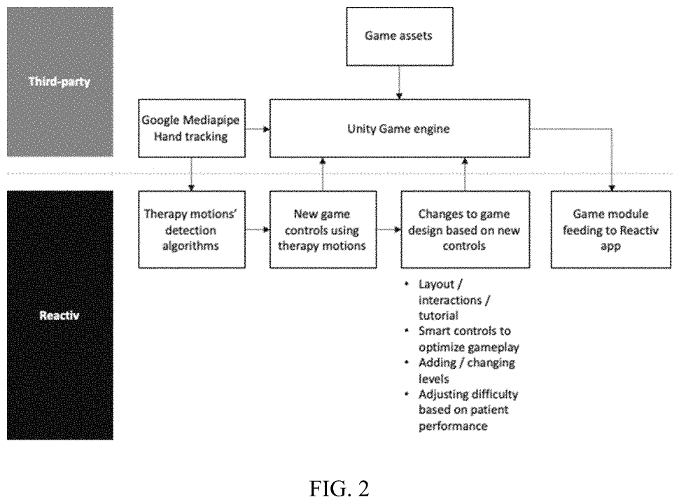

FIG. 2 illustrates how modules, and in particular games, are built. The output of this design and engineering process is a “Game Module.” A Game Module is an activity that a patient can “play” for their rehab, if their therapist thinks it relevant. Other types of modules, such as “Educational Experiences” or “Measurement Tools” are built in a similar way but may not be full games.

### Detection Algorithms

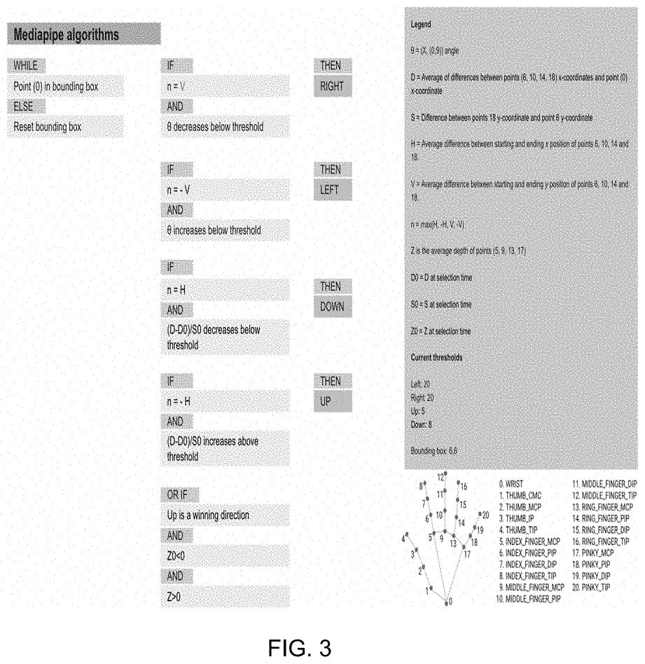

The detection algorithms are designed to detect medically correct motions for Range of Motion exercises. FIG. 3 illustrates one embodiment of detection algorithms for a Candy Game.

### Therapist Control

To tailor the patient's experience to their rehab progression, the platform enables the therapist to adjust the experience settings. In particular, the therapist may:
Enable/Disable specific games and specific motions within games; and
Set the angle threshold needed to complete a given motion.

As the patient's capabilities and range of motion improves, the therapist opens up new experiences and increases the difficulty of the experiences, when the patient is deemed ready.

### Data Collection

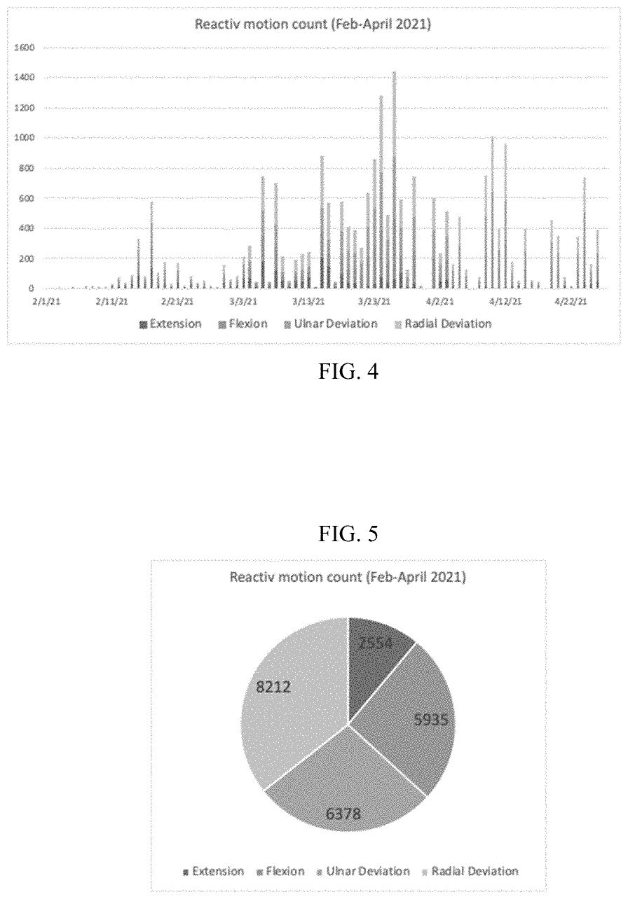

The application disclosed herein may collect data on patient performance in the game. Over an exemplary three months period of time, the activity of users in the Candy Game was as illustrated in FIGS. 4 and 5.

Additional features include collecting data on the patient's performance outside of the games, for example, including a virtual goniometer, a pain scale, and a QuickDASH questionnaire to monitor patients on their Range of Motion, Pain and Functional Capabilities.

1. A physical rehabilitation tool, comprising:
a personal electronic device having a processor, a camera and a display screen;
the processor programed to illustrate a game module on the display screen, the game module including at least a first feature and a second feature; and
the camera configured to superimpose an image of a body part over the game module;
wherein movement of the image of the body part from the first feature to the second feature requires the body part to traverse a desired range of motion.
